(19) United States
(12) Patent Application Publication
(10) Pub. No.: US 2022/0395542 A1
(43) Pub. Date: Dec. 15, 2022

(54) Nutritional Compositions for Treating a Clostridium Difficile Infection

(71) Applicant: ABBOTT LABORATORIES, Abbott Park, IL (US)

(72) Inventors: Enrique VÁZQUEZ HERNÁNDEZ, Ogijares (ES); Rachael BUCK, Gahanna, OH (US); JoMay CHOW, Westerville, OH (US); Karen GOEHRING, Columbus, OH (US)

(21) Appl. No.: 17/776,792

(22) PCT Filed: Nov. 19, 2020
(86) PCT No.: PCT/US2020/061194

§ 371 Date: May 13, 2022

Related U.S. Application Data
(60) Provisional application No. 62/937,986, filed on Nov. 20, 2019.

(51) Int. Cl. A61K 35/745; A61K 31/702; A61K 47/36; A61K 35/747; A61K 35/744; A61K 35/741; A61K 36/064; A61P 31/04

(52) U.S. Cl. CPC A61K 35/745; A61K 31/702; A61K 47/36; A61K 35/747; A61K 35/744; A61K 35/741; A61K 36/064; A61P 31/04

(57) ABSTRACT

A nutritional composition comprises fucosylated human milk oligosaccharide and/or sialylated human milk oligosaccha-ride, non-digestible, fermentable polysaccharide, and Bifidobacterium. The nutritional composition is free of short-chain fructooligosac-charide having at least about 50% of molecules with a degree of polymerization of less than about 5. A method of treating a subject at risk of developing a Clostridium difficile infection or a subject having a Clostridium difficile infection comprises administering such a nutritional composition.

### TECHNICAL FIELD

The present disclosure relates to nutritional compositions for treating a subject at risk of developing a Clostridium difficile infection (CDI) or a subject having a CDI. The present disclosure also relates to nutritional compositions and methods for treating a subject at risk of developing a CDI or a subject having a CDI.

### BACKGROUND

Clostridium difficile (CD) is an anaerobic, spore-forming, toxin-producing, gram-positive bacterium that is transmitted among humans via the fecal-oral route. Elderly, immunocompromised individuals whose gut microbiota have been disrupted by antibiotic therapy have the greatest risk of contracting this highly-contagious, life-threatening, and potentially-fatal diarrheal disease. After highly-virulent strains began appearing during the early 2000s, CD emerged as a major, globally-distributed enteric pathogen.

Generally, CD is regarded as a nosocomial pathogen, being hospital-acquired. However, the disease is now appearing more frequently in individuals once considered to be at low risk. These cases tend to occur in younger individuals. Recent antibiotic exposure is an important risk factor in this population, but use of gastric acid suppressants and co-morbidities such as inflammatory bowel disease, chronic kidney disease, immunodeficiency disease, malignant lesions and solid organ transplants have also been associated with CDI.

Currently, the standard treatment for CDI is antibiotics, with vancomycin or fidaxomicin being the most commonly prescribed compounds. Metronidazole is only used if vancomycin or fidaxomicin are not available. Fecal microbiota transplant (FMT) has only been used as a last resort following multiple recurrences. Probiotics have also been used as a preventive treatment with mixed results. Vaccines are not yet available. Within healthcare facilities, preventive measures include judicious use of antibiotics and infection control practices. Use of monoclonal antibodies has also been studied with the aim of improving host resistance to CDI, however this approach has yielded mixed results.

There is a need in the art for better treatment modalities leading to improved health benefits for subjects. The current invention provides nutritional compositions and methods to decrease incidence of infections from CD and improve subject outcomes.

### SUMMARY

In accordance with the present disclosure, a nutritional composition is provided. The nutritional composition comprises fucosylated human milk oligosaccharide and/or a sialylated human milk oligosaccharide, a non-digestible, fermentable polysaccharide, and Bifidobacterium.

The nutritional composition is free of short-chain fructooligosaccharide (scFOS) having at least about 50% of molecules with a degree of polymerization of less than about 5.

In accordance with the present disclosure, a method of treating a subject at risk of developing a CDI or a subject having a CDI is provided. The method comprises administering to a subject a nutritional composition comprising a fucosylated human milk oligosaccharide and/or a sialylated human milk oligosaccharide, a non-digestible, fermentable polysaccharide, and Bifidobacterium. The nutritional composition is free of scFOS having at least about 50% of molecules with a degree of polymerization of less than about 5.

### BRIEF DESCRIPTION OF THE DRAWINGS

FIG. 1 illustrates results from a control (Control A) experiment using an in vitro model of a gastrointestinal (GI) tract assessing CDI and recurrence as described herein;

FIG. 2 illustrates results from a control (Control B) experiment using an in vitro model of a GI tract assessing CDI and recurrence as described herein;

FIG. 3 illustrates results from an experiment using an in vitro model of a GI tract to assess CDI and recurrence after a treatment of specific human milk oligosaccharides (HMOs), corn fiber (fiber 2) and gum arabic (fiber 3), and a probiotic;

FIG. 4 illustrates results from an experiment using an in vitro model of a GI tract assessing CDI and recurrence after a treatment of a probiotic;

FIG. 5 illustrates results from an experiment using an in vitro model of a GI tract assessing CDI and recurrence after a treatment of HMO;

FIG. 6 illustrates results from an experiment using an in vitro model of a GI tract assessing CDI and recurrence after a treatment of scFOS (fiber 1), and fibers 2 and 3;

FIG. 7 illustrates results from an experiment using an in vitro model of a GI tract assessing CDI and recurrence after a treatment of HMO and fibers 2 and 3;

FIG. 8 illustrates results from an experiment using an in vitro model of a GI tract assessing CDI and recurrence after a treatment of HMO, and fibers 1, 2 and 3;

FIG. 9 illustrates results from an experiment using an in vitro model of a GI tract assessing CDI and recurrence after a treatment of fibers 1, 2, 3, and a probiotic;

FIG. 10 illustrates results from an experiment using an in vitro model of a GI tract assessing CDI and recurrence after a treatment of HMO and a probiotic;

FIG. 11 illustrates results from an experiment using an in vitro model of a GI tract assessing CDI and recurrence after a treatment of HMO, fibers 1, 2, 3, and a probiotic; and

FIG. 12 illustrates results from an experiment using an in vitro model of a GI tract assessing CDI and recurrence after a treatment of fibers 2 and 3.

### DETAILED DESCRIPTION

Specific embodiments of the present disclosure will now be described. The invention can, however, be embodied in different forms and should not be construed as limited to the embodiments set forth herein. Rather, these embodiments are provided to illustrate more specific features of certain aspects of the invention to those skilled in the art.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive in a manner similar to the term “comprising” as that term is interpreted when employed as a transitional word in a claim. Furthermore, to the extent that the term “or” is employed (e.g., A or B), it is intended to mean “A or B or both.” When the “only A or B but not both” is intended, then the term “only A or B but not both” will be employed. Thus, use of the term “or” herein is the inclusive, and not the exclusive use. When the term “and” as well as “or” are used together, as in A “and/or” B this indicates A or B as well as A and B.

The nutritional compositions and corresponding methods of making the nutritional compositions of the present disclosure can comprise, consist of, or consist essentially of any of the elements of the disclosure as described herein.

All ranges and parameters, including but not limited to percentages, parts, and ratios disclosed herein are understood to encompass any and all sub-ranges subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein can be performed in any order, unless otherwise specified or clearly implied to the contrary by the context in which the referenced combination is made.

The phrase “degree of polymerization” refers to the number of monomer units, for example monomeric saccharide units, in a molecule. The phrase “having at least about 50% of molecules with a degree of polymerization” from a first number to a second number, as used herein, unless otherwise specified, refers to an ingredient having multiple molecules composed of varying numbers of monomeric saccharide units, with at least about half of the molecules having a number of saccharide units falling within a range from the first number to the second number.

The term “non-digestible, fermentable polysaccharide” as used herein, unless otherwise specified, refers to a polymeric carbohydrate molecule having at least about 50% of molecules with a degree of polymerization greater than or equal to 10, that resists digestion in the small intestine and is completely or partially fermented in the large intestine. Non-limiting examples of non-digestible, fermentable polysaccharides include inulin, gum arabic, and corn fiber. Specific embodiments of nutritional compositions described herein contain inulin, gum arabic, and/or corn fiber.

The term “oligosaccharide” as used herein, unless otherwise specified, refers to a carbohydrate molecule having at least about 50% of molecules with a degree of polymerization from 2 to 9.

The term “scFOS” as used herein, unless otherwise specified, refers to short-chain fructooligosaccharides, and more specifically, to carbohydrate molecules composed of fructose molecules, wherein the scFOS has at least about 50% of molecules with a degree of polymerization from 1 to 5.

The term “human milk oligosaccharide” (HMO) as used herein, unless otherwise specified, refers to an oligosaccharide derived from milk secreted by a human, and also refers to a milk oligosaccharide from a non-human mammal including, but not limited to bovine, ovine, porcine, and/or caprine species. When referring to non-human mammals, the obtained milk oligosaccharide are deemed HMOs if the milk oligosaccharides are equivalent in structure and/or function, or are structural and/or chemical analogs of milk oligosaccharide secreted by a human. In additional embodiments, the HMOs are produced via microbial fermentation, enzymatic processes, chemical synthesis, or a combination thereof.

The term “prebiotic” as used herein, unless otherwise specified, refers to a non-digestible food ingredient that beneficially affects a subject by selectively stimulating the growth and/or activity of bacteria in a subject's gastrointestinal (GI) tract. Non-digestible, fermentable polysaccharides are examples of prebiotics.

The term “probiotic” as used herein, unless otherwise specified, refers to a microorganism such as a bacteria or yeast that survives the digestive process to confer a health benefit on the host. Examples of probiotics that can be included in nutritional compositions described herein, either alone or in combination, include Bifidobacterium (B.), such as B. breve M-16V, B. infantis Bb02, B. infantis M-63, B. infantis 35624, B. lactis Bb12, B. lactis HN019, B. lactis Bi07, B. bifidum, B. longum BB536, B. longum AH1205, B. longum AH1206, and B. animalis, and Lactobacillus (L.), such as L. rhamnosus GG, L. rhamnosus HN001, L. acidophilus LA-5, L. acidophilus NCFM, L. fermentum CECT5716, L. reuteri ATCC55730, L. reuteri ATCC PTA-6475, and L. reuteri DSM 17938, Streptococcus thermophilus Th4, Akkermansia, Bacteroides, Enterococcus, Eubacterium, Fecalibacterium, Roseburia, and/or Saccharomyces.

The phrase “tube feeding nutritional composition” as used herein refers to a nutritional composition that is formulated to be administered to a subject's gastrointestinal system via a feeding tube. Examples of feeding tube arrangements that can be used to administer the tube feeding nutritional composition include, but are not limited to, a gastric tube, a nasogastric tube, a jejunal tube, and a gastro-jejunal tube.

The term “free of” as used herein, unless otherwise specified, refers to a substance that contains less about 1 wt % of such ingredient. Specific embodiments contain less than 0.5 wt % of the ingredient or less than 0.25 wt % of the ingredient. Specific embodiments contain less than 0.1 wt % of the ingredient, or no measureable amount of the ingredient, i.e., 0 wt %.

Specific embodiments of nutritional compositions provided herein are in the form of powder, liquids (e.g., reconstituted powder, beverage, oil and/or water drops, syrup), solids (e.g., bar, tablet, capsule, candy or gum), or semi-solids (pudding, paste, or gel) and are, or can be incorporated into, a food or can comprise a dietary supplement.

In specific embodiments, when the nutritional composition is a liquid, a serving ranges from about 1 ml to about 500 ml, including from about 110 ml to about 500 ml, from about 110 ml to about 417 ml, from about 120 ml to about 500 ml, from about 120 ml to about 417 ml, from about 177 ml to about 417 ml, from about 207 ml to about 296 ml, from about 230 m to about 245 ml, from about 110 ml to about 237 ml, from about 120 ml to about 245 ml, from about 110 ml to about 150 ml, and from about 120 ml to about 150 ml. In specific embodiments, the serving is about 1 ml, or about 100 ml, or about 237 ml, or about 500 ml.

In specific embodiments, when the nutritional composition is a liquid, solid, semi-solid, or powder, including when the composition comprises a powder or liquid supplement, the composition provides up to about 500 kcal of energy per serving of the nutritional composition, including from about 20 kcal to about 500 kcal, from about 75 kcal to about 500 kcal, from about 150 kcal to about 500 kcal, from about 250 kcal to about 500 kcal, from about 300 kcal to about 500 kcal, or from about 400 kcal to about 500 kcal per serving as described herein of the nutritional composition.

In specific embodiments, the liquid nutritional composition in has a caloric density of about 0.5 kcal/ml to about 3 kcal/ml. In specific embodiments, the nutritional composition has a caloric density from about 0.5 kcal/ml to about 2.5 kcal/ml, including about 0.5 kcal/ml to about 2 kcal/ml, about 0.5 kcal/ml to about 1.5 kcal/ml, about 0.5 kcal/ml to about 1 kcal/ml, or about 0.5 kcal/ml to about 0.8 kcal/ml. In specific embodiments, the nutritional composition has a caloric density of about 1 kcal/ml to about 3 kcal/ml, including about 1.5 kcal/ml to about 3 kcal/ml, about 2 kcal/ml to about 3 kcal/ml, or about 2.5 kcal/ml to about 3 kcal/ml.

In specific embodiments, the nutritional composition is in a liquid form, and has a pH of about 6 to about 8, or is in a powder form and, upon reconstitution with water, forms a liquid having a pH of about 6 to about 8. In specific embodiments the nutritional composition is a powder or liquid supplement having a pH of about 6 to about 8.

In specific embodiments, when the nutritional composition is a liquid, solid, semi-solid, or powder, including when the nutritional composition comprises a powder or liquid supplement, the composition includes a protein, a carbohydrate, and/or a fat. A wide variety of sources and types of protein, carbohydrate, and fat can be used in embodiments of nutritional compositions described herein. In specific embodiments, when the when the nutritional composition comprises a powder or liquid supplement, the composition includes zero, one, two, or three of: a protein, a carbohydrate, and/or a fat.

In specific embodiments of the nutritional composition as described herein, when the nutritional composition comprises a liquid, solid, semi-solid, or powder, including but not limited to when the composition comprises a powder or liquid supplement, the composition comprises protein comprising from about 0 wt % to about 30 wt % of the nutritional composition. In specific embodiments, the protein comprises from about 0.1 wt % to about 25 wt % of the nutritional composition, including about 0.5 wt % to about 20 wt %, about 1 wt % to about 15 wt %, about 1 wt % to about 10 wt %, about 5 wt % to about 10 wt %, or about 10 wt % to about 20 wt % of the nutritional composition. In specific embodiments, the protein comprises from about 1 wt % to about 5 wt % of the nutritional composition. In additional, specific embodiments, the protein comprises from about 20 wt % to about 30 wt % of the nutritional composition.

In specific embodiments of the nutritional composition, one or more sources of protein are used in the nutritional composition as described herein, when the nutritional composition comprises a liquid, solid, semi-solid, or powder, including but not limited to when the composition comprises a powder or liquid supplement. For example, the source of protein can include, but is not limited to, intact, hydrolyzed, and/or partially hydrolyzed protein, which can be derived from a suitable source such as milk (e.g., casein, whey), animal (e.g., meat, fish), cereal (e.g., rice, corn), vegetable (e.g., soy, pea), and combinations thereof. The source of protein can also include a mixture of amino acids (often described as free amino acids) known for use in nutritional products or a combination of such amino acids with the intact, hydrolyzed, and/or partially hydrolyzed proteins described herein. The amino acids can be naturally occurring or synthetic amino acids.

More particular examples of sources of protein used in specific embodiments of the nutritional composition, when the nutritional composition comprises a liquid, solid, semi-solid, or powder, including but not limited to when the composition comprises a powder or liquid supplement, include, but are not limited to, whey protein concentrate, whey protein isolate, whey protein hydrolysate, acid casein, sodium caseinate, calcium caseinate, potassium caseinate, casein hydrolysate, milk protein concentrate, milk protein isolate, milk protein hydrolysate, nonfat dry milk, condensed skim milk, soy protein concentrate, soy protein isolate, soy protein hydrolysate, pea protein concentrate, pea protein isolate, pea protein hydrolysate, collagen protein, collagen protein isolate, rice protein, potato protein, earthworm protein, insect protein, and combinations thereof.

In specific embodiments of the nutritional composition, when the nutritional composition comprises a liquid, solid, semi-solid, or a powder, including but not limited to when the composition comprises a powder or liquid supplement, the composition comprises carbohydrate in an amount from about 0 wt % to about 75 wt % of the nutritional composition. In specific embodiments, the carbohydrate is present in an amount from about 0.1 wt % to about 70 wt % of the nutritional composition, including about 0.5 wt % to about 65 wt %, about 10 wt % to about 65 wt %, about 20 wt % to about 65 wt %, about 30 wt % to about 65 wt %, about 40 wt % to about 65 wt %, or about 15 wt % to about 25 wt % of the nutritional composition.

Carbohydrates in specific embodiments of a nutritional composition as described herein, when the nutritional composition comprises a liquid, solid, semi-solid, or powder, including but not limited to when the composition comprises a powder or liquid supplement, comprise simple, complex, or a combination thereof. Non-limiting examples of a source of carbohydrate suitable for use in specific embodiments of a nutritional composition described herein include HMOs, maltodextrin, hydrolyzed starch, glucose polymers, corn syrup, corn syrup solids, rice-derived carbohydrates, sucrose, glucose, lactose, honey, sugar alcohols, isomaltulose, sucromalt, pullulan, potato starch, galactooligosaccharides, oat fiber, soy fiber, corn fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, and combinations thereof.

In specific embodiments, when the nutritional composition comprises a liquid, solid, semi-solid, or powder, including but not limited to when the composition comprises a powder or liquid supplement, the composition comprises fat at from about 0 wt % to about 30 wt % of the nutritional composition. In certain specific embodiments, the fat comprises from about 0.1 wt % to about 30 wt % of the nutritional composition, including about 0.5 wt % to about 30 wt %, about 10 wt % to about 30 wt %, about 15 wt % to about 30 wt %, about 20 wt % to about 25 wt %, about 5 wt % to about 10 wt %, or about 10 wt % to about 20 wt % of the nutritional composition.

Specific embodiments of the invention as described herein, when the nutritional composition comprises a liquid, solid, semi-solid, or powder, including but not limited to when the composition comprises a powder or liquid supplement, the composition comprises one or more components to modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth.

Specific embodiments of a nutritional composition as described herein, when the nutritional composition comprises a liquid, solid, semi-solid, or powder, including but not limited to when the composition comprises a powder or liquid supplement, the composition comprises vitamins and/or related nutrients, non-limiting examples of which include vitamin A, vitamin B12, vitamin C, vitamin D, vitamin K, thiamine, riboflavin, pyridoxine, niacin, folic acid, pantothenic acid, biotin, choline, inositol, salts and derivatives thereof, and combinations thereof.

In specific embodiments of a nutritional composition as described herein, when the nutritional composition is a liquid, solid, semi-solid, or powder, including when the composition comprises a powder or liquid supplement, the composition comprises minerals, non-limiting examples of which include calcium, phosphorus, magnesium, zinc, manganese, sodium, potassium, molybdenum, chromium, iron, copper, chloride, and combinations thereof.

CD is known as an opportunistic pathogen. Following disruption of the gut microbiota, CD colonizes the colon and secretes two toxins: toxin A and toxin B. These toxins cause severe diarrhea and colonic inflammation which may progress into life-threatening pseudomembranous colitis. Patients who experience one episode have a 20% chance of recurrence.

In specific embodiments, wherein the invention provides for nutritional compositions as described herein comprising a liquid, solid, semi-solid, or powder, including when the composition comprises a powder or liquid supplement, the composition comprises a unique combination of HMOs, fermentable fibers, and a probiotic, and presents a simple, inexpensive, and safe method of reducing colonization by CD, thereby treating CDI and/or reducing the likelihood of a CDI infection. Specific embodiments of treatment methods employ nutritional compositions as described herein alone. Other, specific embodiments of treatment methods employ the nutritional compositions in combination with one or more other treatments for CDI.

Specific embodiments of powdered nutritional compositions comprise, by weight of the nutritional composition, fucosylated HMO in a range from about 0.01 wt % to about 10 wt % and/or sialylated HMO in a range from about 0.01 wt % to about 10 wt %. In a more specific embodiment both the fucosylated HMO and the sialylated HMO are included in the nutritional compositions. Specific embodiments of the nutritional compositions also comprise non-digestible, fermentable polysaccharide in a range from about 0.1 wt % to about 25 wt % and Bifidobacterium in a range from about 10 cfu/g to about 109 cfu/g.

In additional embodiments, the powdered nutritional composition comprises fucosylated HMO and/or sialylated HMO, each in a range up to about 15% of the powdered nutritional composition, including from about 0.01% to about 15%, or from about 0.04 to about 14.9%, or from about 0.1 wt % to about 8 wt %. In yet alternative embodiments, the powdered nutritional composition comprises fucosylated HMO and/or sialylated HMO, each in a range from about 1 wt % to about 6 wt %. In more specific embodiments of each such composition both the fucosylated HMO and the sialylated HMO are included.

In additional embodiments, the powdered nutritional composition comprises non-digestible, fermentable polysaccharide in a range from about from about 0.1 wt % to about 20 wt %, or from about 0.25% to about 10%, or from about 0.3% to about 8%, or from about 0.5% to about 5%, or from about 5% to about 15%.

In additional embodiments, the powdered nutritional composition comprises up to 109 cfu/g of a probiotic or combination of probiotics as described herein, including in a range from about 10 cfu/g to about 109 cfu/g, or from about 10² cfu/g to about 107 cfu/g, or from about 10³cfu/g to about 10⁶ cfu/g, or from about 104 cfu/g to about 10⁶ cfu/g. For example, in specific embodiments, the powdered nutritional composition comprises up to 109 cfu/g of Bifidobacterium, including in a range from about 10 cfu/g to about 109 cfu/g, or from about 10² cfu/g to about 107 cfu/g, or from about 10³cfu/g to about 10⁶ cfu/g, or from about 104 cfu/g to about 10⁶ cfu/g.

In specific embodiments, the nutritional composition comprises a supplement comprising fucosylated human milk oligosaccharide and/or the sialylated human milk oligosaccharide up to about 50 wt % of the supplement, at least 10 wt % of non-digestible, fermentable polysaccharide, and from about 5×10² to about 5×10⁸ cfu/g of at least one probiotic as described herein, such as Bifidobacterium, and the supplement is free of short-chain fructooligosaccharide having at least about 50% of molecules with a degree of polymerization of less than about 5. In specific embodiments, the supplement comprises a total weight of up to about 50 grams, including a range such as from about 0 grams to about 20 grams, and about 5 grams to about 30 grams.

Yet more specific examples of powdered nutritional compositions of the current invention comprise from about 0.01 wt % to about 10 wt % each of 2′-Fucosyllactose (2′-FL), 6′-Sialyllactose (6′-SL), corn fiber, and gum arabic, and from about 10 to about 109 cfu/ml of Bifidobacterium. Specific embodiments additionally contain 2′FL, 3′-Fucosyllactose (3′FL), and/or Lacto-N-Tetraose (LNT), which are neutral HMOs. Example acidic HMOs of the composition are 3′-Sialyllactose (3′SL), and 6′-SL.

Yet more specific examples of powdered nutritional compositions of the current invention comprise from about 0.01 wt % to about 15 wt % of powder each of 2-FL, 6′SL, corn fiber, and gum arabic, and from about 10 to about 109 cfu/g of Bifidobacterium. Additional embodiments comprise fiber from about 1 wt % to about 10 wt %. Specific embodiments of the powdered nutritional compositions comprise up to about 15 wt % of corn fiber, or from about 1 wt % to about 10 wt % corn fiber, or from about 3 wt % to about 6 wt % corn fiber.

Additional, specific embodiments of liquid nutritional compositions comprise fucosylated HMO in a range from about 0.01 wt % to about 20 wt % and/or the sialylated HMO in a range from about 0.01 wt % to about 20 wt %. Additional, specific embodiments of liquid nutritional compositions comprise fucosylated HMO and/or the sialylated HMO each in a range from about 0.01 wt % to about 20 wt %, or from about 1.0 wt % to about 10.0 wt %, or from about 0.01 wt % to about 5 wt %.

Specific embodiments of the liquid nutritional composition also comprises non-digestible, fermentable polysaccharide in a range from about 0.1 wt % to about 5 wt %, and Bifidobacterium in a range from about 10 cfu/ml to about 109cfu/ml. In a specific embodiment, the compositions include both fucosylated HMO and sialylated HMO.

In alternative embodiments, the liquid nutritional composition comprises fucosylated HMO and/or sialylated HMO each in a range from about 0.05 wt % to about 1.5 wt %. In yet alternative embodiments, the liquid nutritional composition comprises fucosylated HMO and/or sialylated HMO each in a range from about 0.1 wt % to about 1 wt %.

In additional embodiments, the liquid nutritional composition comprises non-digestible, fermentable polysaccharide in a range from about 0.1 wt % to about 5 wt %, or from about 0.5 wt % to about 4 wt %, or from about 1 wt % to about 4 wt %. In specific embodiments, the liquid nutritional composition comprises non-digestible, fermentable polysaccharide comprising corn fiber, in a range from about 0.1 wt % to about 5 wt %, or from about 0.5 wt % to about 4 wt %, or from about 1 wt % to about 4 wt %, or from about 1.2 wt % to about 4 wt %.

In additional embodiments, the liquid nutritional composition comprises a probiotic or combination of probiotics as described herein in a range from about 10 cfu/ml to about 109 cfu/ml, or from about 10² cfu/ml to about 107 cfu/ml, or from about 104 cfu/ml to about 10⁶ cfu/ml. For example, in specific embodiments, the liquid nutritional composition comprises Bifidobacterium in a range from about 10 cfu/ml to about 109 cfu/ml, or from about 10² cfu/ml to about 107 cfu/ml, or from about 104 cfu/ml to about 10⁶ cfu/ml. In additional embodiments, the liquid nutritional composition comprises up to about 4×107 cfu/ml of a probiotic or combination of probiotics including in a range from about 4×101 cfu/ml to about 4×107 cfu/ml. For example, in specific embodiments, the liquid nutritional composition comprises up to about 4×107 cfu/ml of Bifidobacterium including in a range from about 4×101 cfu/ml to about 4×107 cfu/ml.

In specific embodiments of the nutritional composition, when the nutritional composition comprises a liquid, solid, semi-solid, or a powder, including but not limited to when the composition comprises a powder or liquid supplement, the composition is free of scFOS having at least about 50% of molecules with a degree of polymerization of less than about 5.

In specific embodiments of nutritional compositions as described herein, when the nutritional compositions are a liquid, solid, semi-solid, or powder, including when the composition comprises a powder or liquid supplement, the composition comprises one or more of: postbiotics (metabolites of probiotics), long chain polyunsaturated fatty acids (Docosahexanoic acid (DHA), arachidonic acid (ARA), docosapentaenoic acid (DPA), eicosapentaenoic acid (EPA), etc.), nucleotides, antioxidants/anti-inflammatory compounds including tocopherols, carotenoids, ascorbate/vitamin C, ascorbyl palmitate, polyphenols (e.g., curcumin), glutathione, and superoxide dismutase (melon), other bioactive factors (e.g., growth hormones, cytokines, Transforming Growth Factor (TGF) alpha or beta) of human and/or bovine milk origin, human milk-derived lipids, free amino acids or peptides (e.g., beta-hydroxy-beta-methylbutyrate (HMB), arginine, leucine, and/or glutamine), lactose, water or fat soluble vitamins, minerals, and trace elements.

In the methods of the invention, subjects at risk for a CDI, having a primary CDI, or having a recurrent CDI are administered a nutritional composition described herein. In specific embodiments, the subject has tested positive for CD toxin A and/or B, and the step of administering the nutritional composition is performed to reduce the risk of clinical symptoms of a CDI such as diarrhea and/or colonic inflammation. In specific embodiments, the nutritional composition is administered to a subject to reduce the risk of a primary infection or a recurrent infection of Clostridium difficile.

In specific methods, the subject is administered an antibiotic and/or a gastric acid supplement prior to, with, or subsequent to administering a nutritional composition described herein. In specific embodiments, the antibiotic and/or a gastric acid supplement is administered to the subject from about 1 to about 5 days prior to the administration of the nutritional composition, or from about 1 to about 7 days prior to the administration of the nutritional composition, or from about 1 to about 14 days prior to the administration of the nutritional composition. In specific embodiments, the antibiotic is vancomycin or fidaxomicin.

Subjects with inflammation and immunodeficiencies are at risk of developing CDI. Therefore, in specific embodiments, subjects administered a nutritional composition described herein according to the inventive methods have inflammatory-bowel disease, chronic kidney-disease, an immunodeficiency disease, a malignant lesion, or have had a solid organ transplant.

In specific embodiments, the nutritional composition is administered to the subject once or multiple times daily for a time period of up to about 10 weeks. In specific cases, the administration is daily for a time period from about 4 weeks to about 7 weeks, or from about 5 to about 6 weeks. In specific embodiments, the nutritional composition is administered to the subject from about 1 to about 6 times per week, or from about 1 to about 5 times per week, or form about 1 to about 4 times per week, or from about 1 to about 3 times per week.

In specific embodiments, the nutritional composition is administered orally or via tube-feeding. In specific embodiments, when the administration is via tube feeding, the tube-feeding is performed through the nose of the subject or directly into the stomach or small intestine of the subject through an incision in the abdomen of the subject.

The following examples demonstrate aspects of the invention.

### Examples

### Model

An adapted version of an in vitro gut digestion and fermentation model (e.g., a SHIME®PathoGut model; ProDigest, Ghent, Belgium) was used to assess potential anti-pathogenic activity of ingredients and combinations of ingredients against C. difficile. The model tested the ingredients and combinations of ingredients in relation to initial CDI and to CDI recurrence, and included a succession of reactors simulating different parts of the gastrointestinal tract.

More specifically, the in vitro gut model used three reactors operating at 37° C. The reactors contained double-jacketed glass vessels connected through peristaltic pumps. The first reactor, which simulated digestion in the stomach and small intestine, followed a fill-and-draw principle, where a defined nutritional medium, and pancreatic and bile liquid, was added three times a day. The medium was composed of complex carbohydrate and protein sources, mucins, and minerals and vitamins. Upon digestion in the first compartment, the slurry was pumped in the proximal colon (PC) (second) reactor where colonic fermentation was initiated. The model also had a distal colon (DC) (third) reactor. The colonic reactors were continuously stirred with constant volume and pH control.

The reactor simulating the PC contained a volume of 500 ml that was kept at a constant pH within about 5.6 to about 5.9. The DC reactor contained a volume of 800 ml that was kept at a constant pH of about 6.6-6.9. Retention time and pH in the PC and DC reactors were set to simulate in vivo conditions in the PC and DC, respectively.

The model utilized seven stages or periods, as shown in Table 1. In the stabilization period, a CD microbial community was established in the PC and DC reactors, with the community varying based on the fact the PC and DC reactor environments are different. The control period was used to establish a baseline in each compartment and reactor for CD levels. During the prevention treatment, ingredients as described in Tables 2 and 3 were administered to the first reactor to see the effect on reducing CD numbers. During clindamycin treatment, the antibiotic clindamycin was added to the DC and PC to induce dysbiosis. In other words, clindamycin treatment resulted in a microbial imbalance increasing the chance that CD could displace other microbial organisms and establish a stable CDI infection. CD spores were administered into the PC at the start and at the end of clindamycin treatment period. During vancomycin treatment, the antibiotic vancomycin was applied to the PC and DC to simulate treatment of the CDI. During the subsequent washout phase, vancomycin administration was stopped, and CD numbers were determined. During the washout period, recurrences occurred with some treatments, and were prevented by other treatments.

TABLE 1

| Stage | Period or | Dura- | |
|---|---|---|---|
| No. | Treatment | tion | Description |
| 1 | Stabilization | 2 weeks | Period after inoculation of the PC and |
| | Period | | DC reactors with a fresh fecal sample |
| | | | allowing microbial communities to |
| | | | establish and differentiate |
| 2 | Control | 1 week | Samples were collected to establish |
| | Period | | baseline parameters for the gastric and |
| | | | small intestine compartments and the |
| | | | PC and DC reactors |
| 3 | Preventive | 1 week | Ingredients and combinations of |
| | Treatment | | ingredients for treatment were |
| | | | administered to the first compartment. |
| | | | Any fiber and/or HMO was administered |
| | | | three times daily with feed. Any |
| | | | probiotic was administered once daily. |
| | | | Administration of fiber, HMO, and/or |
| | | | probiotic continued through the end of |
| | | | Stage 7 |
| 4 | Clindamycin | 1 week | The antibiotic clindamycin was used to |
| | Treatment | | treat the PC and DC reactors at 33.9 |
| | | | mg/L, three times per day for seven |
| | | | days to induce dysbiosis. Exposure to |
| | | | CD was simulated by administration of |
| | | | 10⁷ CFU CD to the PC reactor |
| 5 | CDI | 4 weeks | Clindamycin treatment was stopped and |
| | | | another dose of 10⁷ CFU CD spores were |
| | | | administered to the PC and DC reactors. |
| 6 | Vancomycin | 1 week | The antibiotic vancomycin was used to |
| | Treatment | | treat the reactors at 125 mg/L, three |
| | | | times per day for seven days. |
| 7 | Wash Out | 3 weeks | Vancomycin treatment was stopped, and |
| | | | treatment with test ingredients continued |
| | | | to assess CDI recurrence |

Individual ingredients and combinations of ingredients were used in various treatment compositions in the model to evaluate the ability to reduce CDI incidence and to provide treatment. Table 2 shows the specific oligosaccharides, fibers, and probiotics which were employed in the treatment compositions.

TABLE 2

| Category | Ingredients |
|---|---|
| HMO | 2′-fucosyllactose and 6′-sialyllactose |
| Fibers | 1 = scFOS (NutraFlora ®; Ingredion, Inc., Westchester, |
| | Illinois, minimum of 95% (dry basis) short-chain |
| | fructooligosaccharides consisting of GF2, GF3 and GF4 |
| | molecules) |
| | 2 = corn fiber (Fibersol-2; Archer Daniels Midland, Chicago, |
| | Illinois) |
| | 3 = gum arabic (TIC Gums, Belcamp, Maryland) |
| Probiotic | Bifidobacterium animalis subsp. lactis Bb12 (Chr. Hansen, |
| | Hoersholm, Denmark) |

More specifically, Samples 1-12 as shown in Table 3 were employed. Samples 1 and 2 comprised controls (A and B), while Samples 3-12 employed individual ingredients or various combinations of ingredients as shown to evaluate the ability to reduce CDI incidence and to provide CDI treatment. Probiotic was administered once each day at a level of 533 mg/day from a stock of 10⁹CFU per gram (administration indicated by “X” in Table 3).

TABLE 3

| | | g/L | | | | | | |
|---|---|---|---|---|---|---|---|---|
| | Ingredients | | | | | Gum | | Total |
| Sample | for Treatment | 2′-FL | 6′-SL | scFOS | Fibersol-2 | Arabic | Probiotic | Fiber |
| 1 | Control A | — | — | — | — | — | — | 0 |
| 2 | Control B | — | — | — | — | — | — | 0 |
| 3 | HMO, fibers 2 | 2.5 | 2.5 | — | 2.5 | 2.5 | X | 10 |
| | and 3, and probiotic | | | | | | | |
| 4 | Probiotic | — | — | — | — | — | X | 0 |
| 5 | HMO | 2.5 | 2.5 | — | — | — | — | 5 |
| 6 | Fibers 1, 2, and 3 | — | — | 1.67 | 1.67 | 1.67 | — | 5 |
| 7 | HMO and | 2.5 | 2.5 | | 2.5 | 2.5 | — | 10 |
| | fibers 2 and 3 | | | | | | | |
| 8 | HMO and | 2.5 | 2.5 | 1.67 | 1.67 | 1.67 | — | 10 |
| | fibers 1, 2 and 3 | | | | | | | |
| 9 | Fibers 1, 2, 3, | — | — | 1.67 | 1.67 | 1.67 | X | 5 |
| | and probiotic | | | | | | | |
| 10 | HMO and | 2.5 | 2.5 | — | — | — | X | 5 |
| | probiotic | | | | | | | |
| 11 | HMO, fibers 1, | 2.5 | 2.5 | 1.67 | 1.67 | 1.67 | X | 10 |
| | 2, 3, and probiotic | | | | | | | |
| 12 | Fibers 2 and 3 | — | — | — | 5 | 5 | — | 10 |

### Assessment

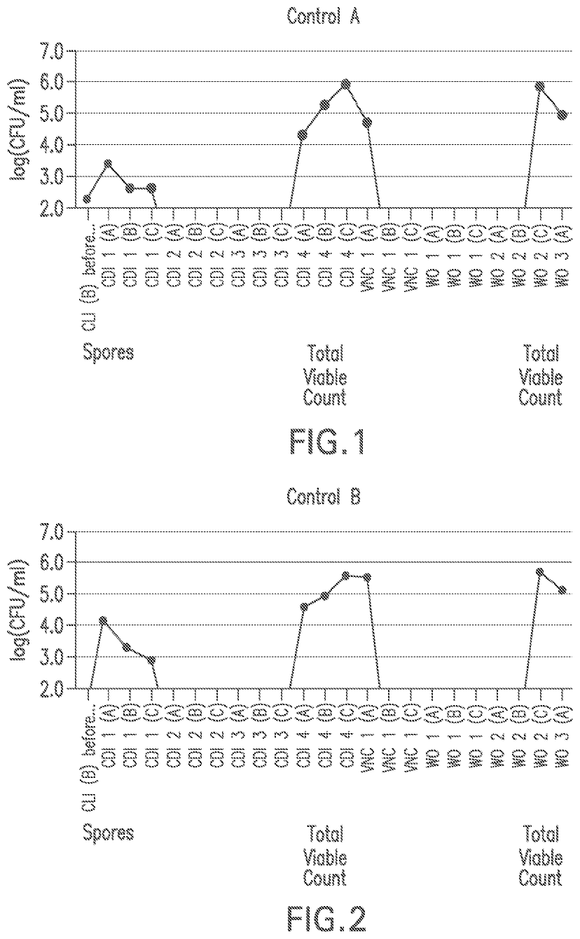

The results are shown in FIGS. 1-12. For each of FIGS. 1-12, the x-axis shows a CDI stage, vancomycin stage (VNC), and a wash out stage (WO) in accord with Stages 5-7 of Table 1. The numbers 1-4 (i.e., of CDI 1, CDI 2, etc.) represent the week of a given stage, and A, B, and C represent respective samples taken within the indicated week of the indicated stage. The CDI stage for the x-axis starts at the end of the clindamycin administration, where dysbiosis is induced to establish a CDI. The CDI stage is 4 weeks long (CDI 1 to CDI 4), the VNC stage is 1 week long (VNC 1), and the wash out stage is 3 weeks long (WO 1 to WO 3).

The y-axis shows a concentration of spores in the CDI week 1 or total viable count (TVC) in the latter weeks/stages. CD spores were added to obtain a resulting spore concentration of about 4.6 log CFU spores/ml inside the PC reactor. This level was reached by adding 10′ CFU of CD spores in 2 ml of solution to 500 ml in the PC reactor, so that the spore stock was diluted with a factor of 250, resulting in a concentration of about 4.6 log CFU spores /ml inside the PC reactor.

Spore counts were obtained after pre-treatment of the sample with ethanol. Ethanol killed vegetative CD cells, while spores were retained. Total viable counts (TVC) came from directly plating a sample on growth medium. As infection in vivo typically occurs in the DC region, the effect of the test ingredients on CDI was assessed by plating dilutions of samples from the DC reactor on a growth medium selective to differentiate for CD.

FIGS. 1 and 2 illustrate the controls A and B, where no treatment ingredients were utilized, and illustrate the baseline CD levels at infection and recurrence. As illustrated in FIG. 1 and FIG. 2, spores were counted for the first week, and infection was seen starting in week 3. After the 4 weeks of CDI, vancomycin was added and this reduced the CD cell count to below detectable levels. However, recurrence of infection as evidenced by TVC was seen in the second wash out week in each of the control experiments as shown in FIGS. 1 and 2.

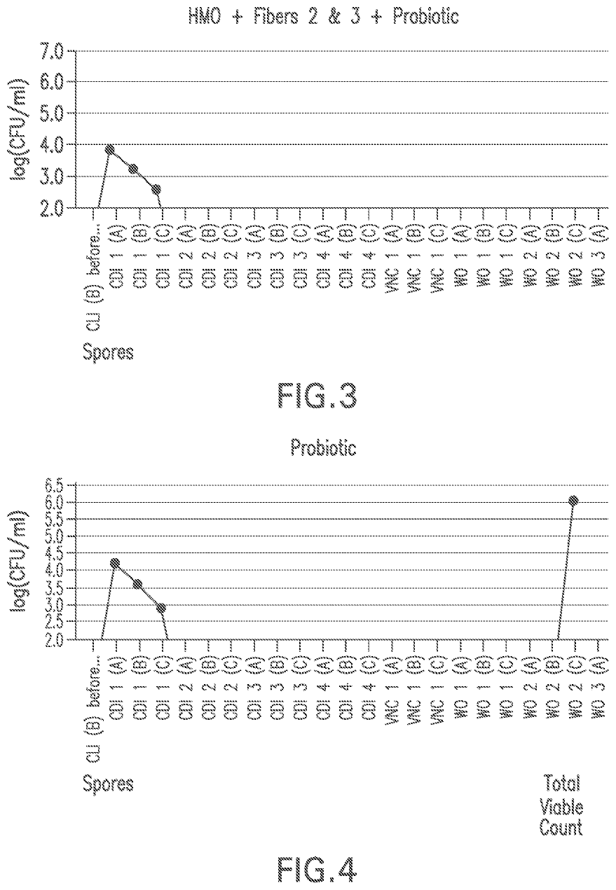

Infection was prevented by two of the treatments, Samples 3 and 4, as shown in FIG. 3 and FIG. 4, where a treatment of HMO, fibers 2 and 3, and probiotic were used in combination, and where probiotic was used individually, respectively. However, only with Sample 3, comprising HMO, fibers 2 and 3, and probiotic together, was recurrence also prevented, as shown in FIG. 3. On the other hand, as shown in FIG. 4, recurrence of infection occurred in the wash out stage with Sample 4, i.e., when treatment of only probiotic was administered.

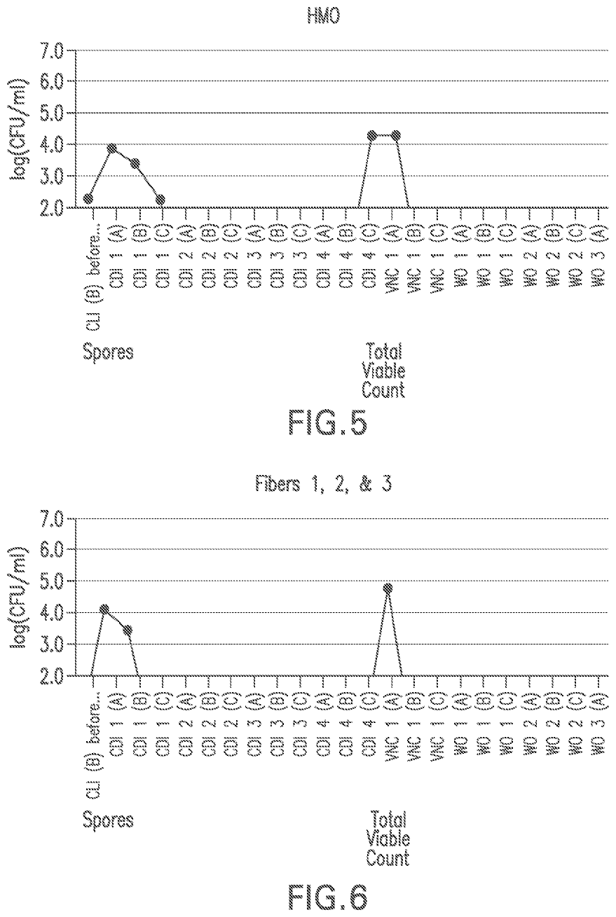

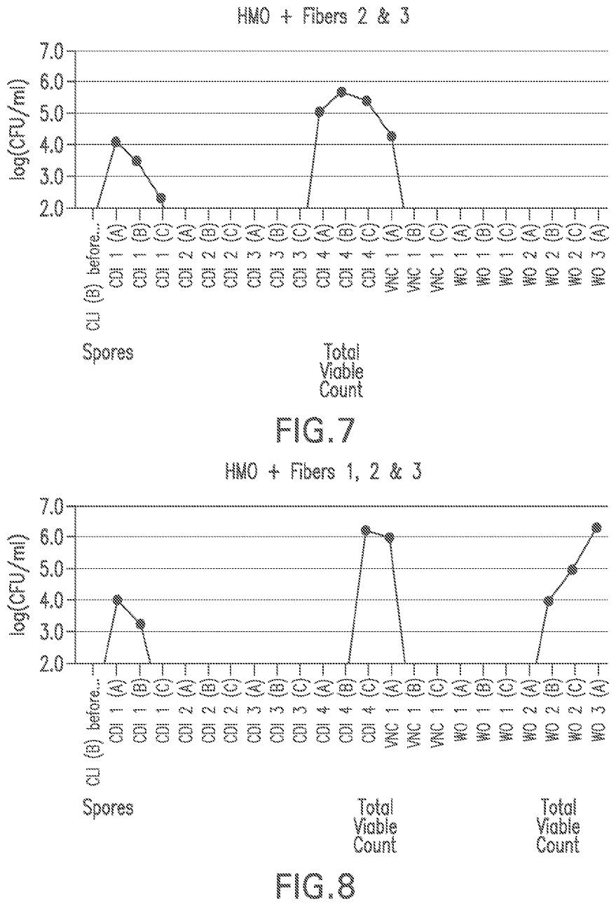

FIGS. 5-7, show treatments using Samples 5-7, respectively, that did not prevent an initial infection but did prevent the recurrence. This included treatment with Sample 5, HMO (FIG. 5), Sample 6, fibers 1, 2 and 3 in combination (FIG. 6), as well as Sample 7, HMO in combination with fibers 2 and 3 (FIG. 7).

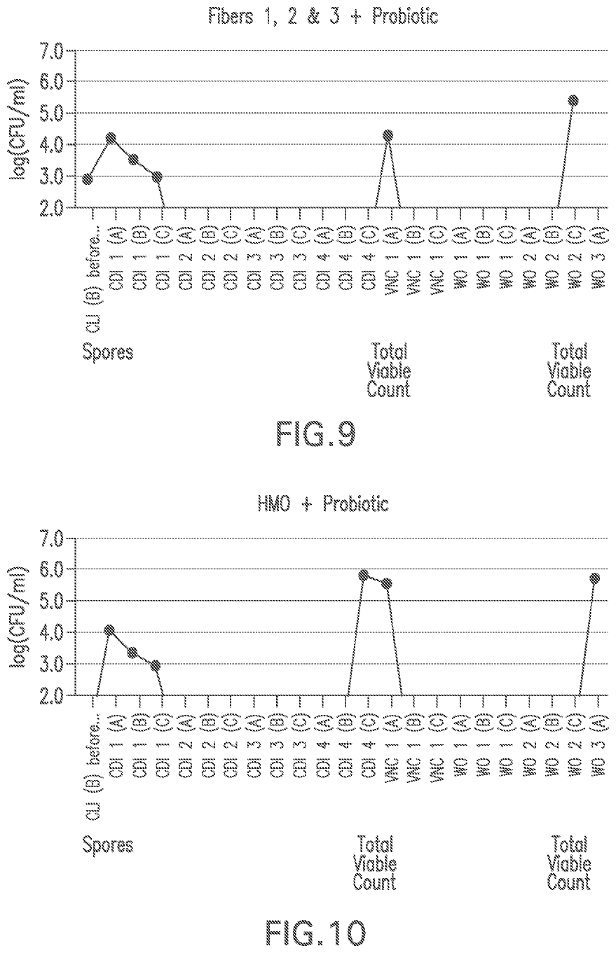

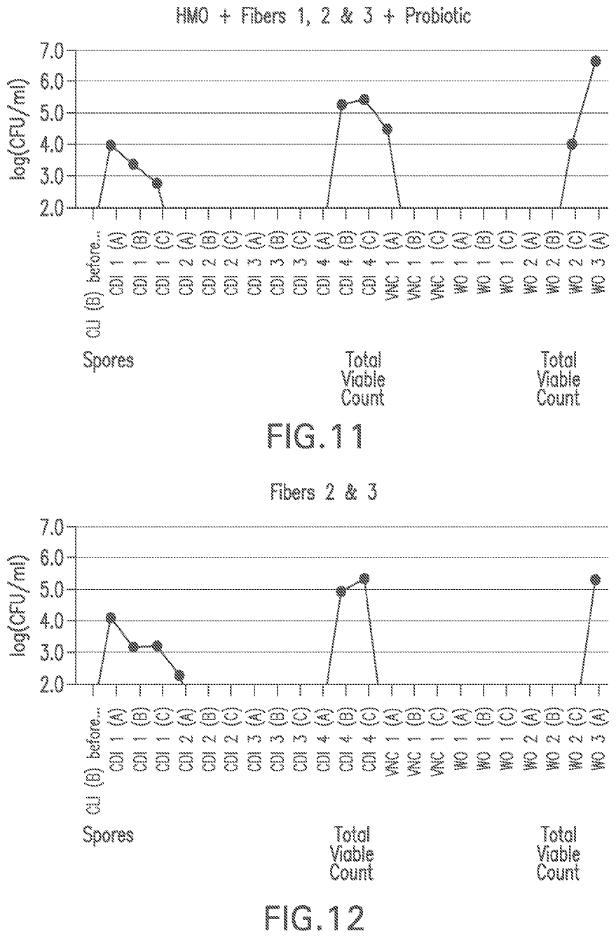

FIGS. 8-12 show treatments that did not prevent the initial infection or recurrence. These treatments included: Sample 8, HMO and fibers 1, 2 and 3 (FIG. 8); Sample 9, fibers 1, 2 and 3, and a probiotic (FIG. 9); Sample 10, HMO and a probiotic (FIG. 10); Sample 11, HMO and fibers 1, 2 and 3, and a probiotic (FIG. 11); and Sample 12, fibers 2 and 3 (FIG. 12).

For the experimental treatments using Samples 5-12, FIGS. 5-12, all of the treatments delayed the time of initial infection except for the treatment of Sample 7, FIG. 7, where HMO, and fibers 2 and 3 in combination led to an initial infection at the same time as controls.

Surprisingly, Sample 11, FIG. 11, includes a combination of HMO, fibers 1, 2 and 3, and a probiotic. This is the same as that of FIG. 3 other than the addition of fiber 1 (scFOS) in Sample 11, FIG. 11. This shows that scFOS negates the effect of the combination of FIG. 3 on CDI.

Examples described herein are exemplary only and are not limiting to the invention defined by the claims.

1. A nutritional composition comprising:
a fucosylated human milk oligosaccharide and/or a sialylated human milk oligosaccharide;
a non-digestible, fermentable polysaccharide; and
Bifzdobacterium;
wherein the nutritional composition is free of short-chain fructooligosaccharide having at least about 50% of molecules with a degree of polymerization of less than about 5.

2. A method of treating a subject at risk of developing a Clostridium difficile infection or a subject having a Clostridium difficile infection, the method comprising: administering to the subject a nutritional composition comprising:
a fucosylated human milk oligosaccharide and/or a sialylated human milk oligosaccharide;
a non-digestible, fermentable polysaccharide; and
Bifzdobacterium;
wherein the nutritional composition is free of short-chain fructooligosaccharide having at least about 50% of molecules with a degree of polymerization of less than about 5.

3. The method of claim 2, wherein the nutritional composition comprises a powdered nutritional composition comprising, by weight of the nutritional composition:
the fucosylated human milk oligosaccharide in a range from about 0.01 wt % to about 10 wt % and/or the sialylated human milk oligosaccharide in a range from about 0.01 wt % to about 10 wt %;
the non-digestible, fermentable polysaccharide in a range from about 0.1 wt % to about 25 wt %; and
the Bifidobacterium in a range from about 10 cfu/g to about 10⁹ cfu/g.

4. The method of claim 2, wherein the nutritional composition comprises a liquid nutritional composition comprising:
the fucosylated human milk oligosaccharide in a range from about 0.01 wt % to about 2 wt % and/or the sialylated human milk oligosaccharide in a range from about 0.01 wt % to about 2 wt %;
the non-digestible, fermentable polysaccharide in a range from about 0.1 wt % to about 5 wt %; and
the Bifidobacterium in a range from about 10 cfu/ml to about 10⁹ cfu/ml.

5. The nutritional composition of claim 1, wherein the nutritional composition comprises a supplement comprising:
the fucosylated human milk oligosaccharide and/or the sialylated human milk oligosaccharide in an amount of up to about 50 wt % of the supplement;
at least 10 wt % of the non-digestible, fermentable polysaccharide, based on the weight of the supplement; and
from about 5×10² to about 5×10⁸ cfu/g of the Bifidobacterium.

6. The method of claim 2, wherein the nutritional composition comprises the fucosylated human milk oligosaccharide and the sialylated human milk oligosaccharide.

7. The method of claim 2, wherein the fucosylated human milk oligosaccharide is 2′-fucosyllactose, and the sialylated human milk oligosaccharide is 6′-sialyllactose.

8. The method of claim 2, wherein the non-digestible, fermentable polysaccharide comprises gum arabic, corn fiber, or a combination of the gum arabic and the corn fiber.

9. The method of claim 2, wherein the Bifidobacterium comprises Bifidobacterium animalis, Bifdobacterium breve, Bifidobacterium longum, Bifidobacterium infantis, and/or Bifdobacterium bifidum.

10. The method of claim 2, wherein the nutritional composition further comprises Lactobacillus (L.) rhamnosus GG, L. rhamnosus HN001, L. acidophilus LA-5, L. acidophilus NCFM, L. fermentum CECT5716, L. reuteri ATCC55730, L. reuteri ATCC PTA-6475, L. reuteri DSM 17938, Streptococcus thermophilus Th4, Akkermansia, Bacteroides, Enterococcus, Enterococcus, Eubacterium, Fecalibacterium, Roseburia, and/or Saccharomyces.

11. The method of claim 2, wherein the nutritional composition further comprises an anti-inflammatory compound.

12. The method of claim 2, wherein the subject has a primary infection of Clostridium difficile.

13. The method of claim 2, wherein the subject has a recurrent infection of Clostridium difficile.

14. The method of claim 2, wherein the subject has tested positive for Clostridium difficile toxin A and/or B, and the step of administering the nutritional composition is performed to reduce the risk of diarrhea or colonic inflammation.

15. The method of claim 2, wherein the subject at risk for a Clostridium difficile infection was administered an antibiotic and/or a gastric acid supplement from about 1 to about 7 days prior to the administration of the nutritional composition.

16. The method of claim 14, wherein the subject was administered an antibiotic, and the antibiotic was vancomycin or fidaxomicin.

17. The method of claim 2, wherein the subject has inflammatory-bowel disease, chronic kidney-disease, an immunodeficiency disease, a malignant lesion, or has had a solid organ transplant.

18. The method of claim 4, wherein about 237 ml of the liquid nutritional composition is administered to the subject.

19. The method of claim 2, wherein the nutritional composition is administered to the subject daily for a time period of from about 4 weeks to about 7 weeks.

20. The method of claim 2, wherein the step of administering the nutritional composition to the subject comprises administering the nutritional composition via tube-feeding.

21. The method of claim 2, wherein the step of administering the nutritional composition is performed to reduce the risk of the primary infection of Clostridium difficile.

22. The method of claim 2, wherein the step of administering the nutritional composition is performed to reduce the risk of the recurrent infection of Clostridium difficile.
